(19) United States
(12) Patent Application Publication
(10) Pub. No.: US 2006/0293598 A1
(43) Pub. Date: Dec. 28, 2006

(54) Motion-tracking improvements for hifu ultrasound therapy

(75) Inventor: John D Fraser, Woodinville, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., Eindhoven (NL)

(21) Appl. No.: 10/551,430

(22) PCT Filed: Feb. 23, 2004
(86) PCT No.: PCT/IB04/00505

§ 371 Date: Aug. 24, 2005

Related U.S. Application Data
(60) Provisional application No. 60/451,148, filed on Feb. 28, 2003.

(51) Int. Cl. A61B 8/00

(52) U.S. Cl. 600/439

(57) ABSTRACT

High intensity focused ultrasound (HIFU) for medically treating tumors is automatically administered under robotic control in dosage intervals that alternate with ultrasonic imaging intervals. The HIFU transmitter is re-aimed for each dosage to compensate for motion of the tumor due to heart beats and other events.

The present invention relates to high intensity focused ultrasound (HIFU) medical treatment. More specifically, the present invention relates to automatic administration of HIFU dosage that compensates for motion of tissue being treated.

High intensity focused ultrasound (HIFU) is emerging as a modality for use in medical treatment of tumors, as an alternative to more invasive procedures such as surgery. Sound waves of high intensity are sharply focused on one spot at a time to kill the body tissue at that point, before repeating the process for a further point on the tumor tissue to undergo treatment.

Cavitation is a process by which bubbles form and collapse violently in a fluid through which high intensity sound or ultrasound is propagating. It is a pressure-related phenomenon. HIFU can also cause thermal effects including evolution of dissolved air from body fluid, thermal cooking, and boiling of water in the body fluid. Either cavitation or thermal effects can be used to kill tissue. The air bubbles which evolve can be used to monitor the location of the heated region during heating, and may act as a temperature indicator. They have also been used to form a barrier to deeper penetration of the sound beam.

At relatively lower HIFU intensities, the tissue under treatment is merely heated therapeutically but not destroyed.

Magnetic resonance imaging (MRI) or X-ray CT imaging is typically used, preparatory to the treatment, to render a 3 dimensional (3-D) image of the tumor on a display screen. During treatment, the treatment beam is moved within the visualized area under manual-visual control, point by point, stopping at each point to deliver a HIFU dose.

Effective use of cavitation or cooking generally requires between 10 seconds and a minute of HIFU treatment at each spot. The tumor might, if located in the patient's torso, move synchronously with the patient's respiration and/or heart beat. The liver, for example, is near the heart and lungs and will move in response to their movement.

Under current practice, a patient is anesthetized continuously during the HIFU treatment, and the anesthetist stops the patient's breathing during delivery the HIFU dose and restarts the breathing afterwards. Typically, the physician then designates on the display screen another spot for treatment, and, after the anesthetist has again paused the patient's breathing, delivers another dose of HIFU. This regime of starting and stopping respiration is repeated for each spot, with imaging continuing point-by-point or being performed infrequently, until a treatment volume, which includes the tumor and typically some surrounding tissue, is completed, generally over a several hour period. Conventional HIFU treatment methodology is therefore tedious, time-consuming and potentially error-inducing. In particular, the physician is prone to errors in keeping track of what parts of the treatment volume have already been completed and which parts remain to be treated.

Furthermore, an MRI apparatus is usually very expensive, typically costing from one-half to two million dollars, and exposure to X-Rays can entail health risks.

There, consequently, exists a need to make HIFU treatment quicker, safer, and more cost-effective.

An object of the present invention is to overcome the above-mentioned disadvantages of the prior art by providing an apparatus and method for HIFU treatment that is performed under automatic processor control and without the need for user intervention.

An alternative object of the present invention is to provide HIFU treatment that can be carried to completion in a shorter period of time.

Another object of the present invention is to provide HIFU that operates in conjunction with relatively cost-effective ultrasonic imaging.

A yet further object of the present invention is to provide a HIFU treatment scheme that avoids excessive anesthetic interventions and consequent risks to the patient.

In the present invention, a HIFU transmitter and an ultrasonic imaging transceiver are aimed concurrently at a treatment point in the body of a patient and are operated in rapid alternation. If, through comparing images, a processor detects that the treatment volume has moved, the transmitter is immediately re-aimed robotically to compensate for the motion, thereby tracking the treatment point. When HIFU dosage is completed for one point, the processor shifts application to the next point, and so on, until the last point in a 3-D raster scan of the whole treatment volume has been completed. The motion-tracking is preferably aided by ultrasonically high-contrast markers or marking points that are disposed in and around the treatment volume in a preparatory phase that precedes treatment.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

In the drawings, where numbering of like functions is maintained throughout the views:

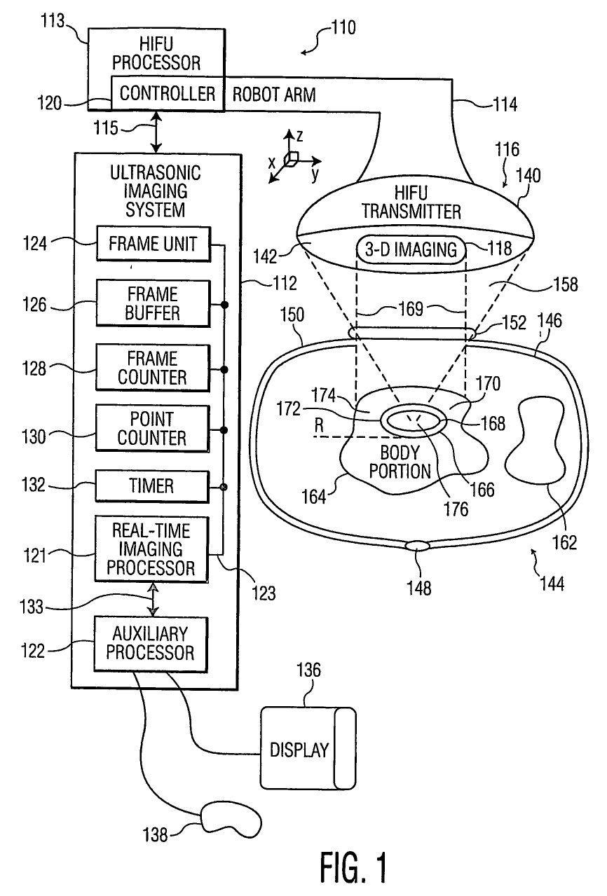

FIG. 1 shows diagrammatically an example of a HIFU apparatus according to the present invention;

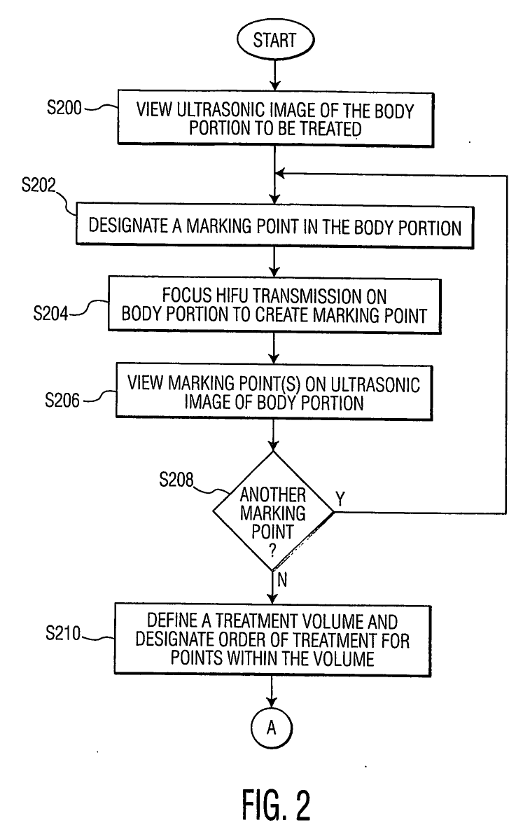

FIG. 2 shows a flow diagram of an exemplary preparatory phase of a method of HIFU treatment according to the present invention;

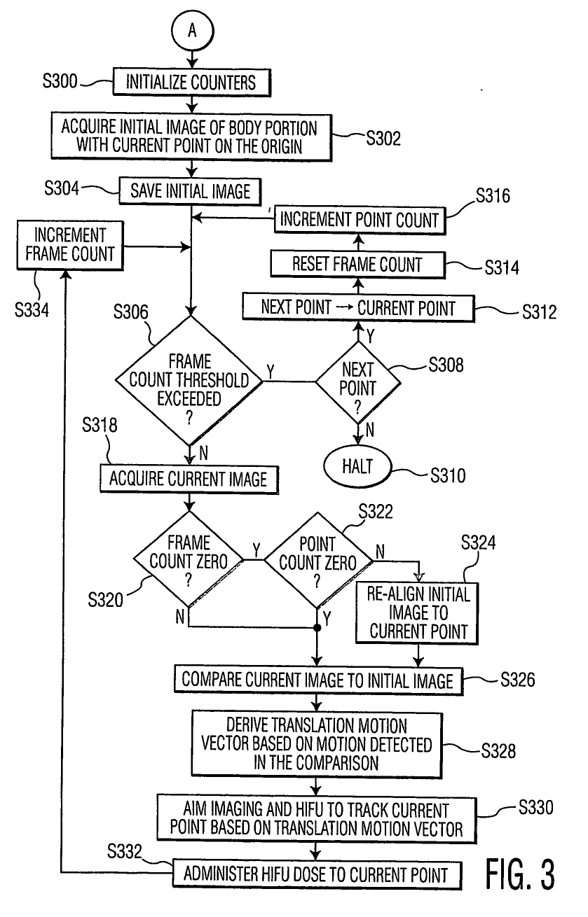

FIG. 3 shows a flow diagram of a first embodiment of an exemplary operational phase of a method of HIFU treatment according to the present invention; and

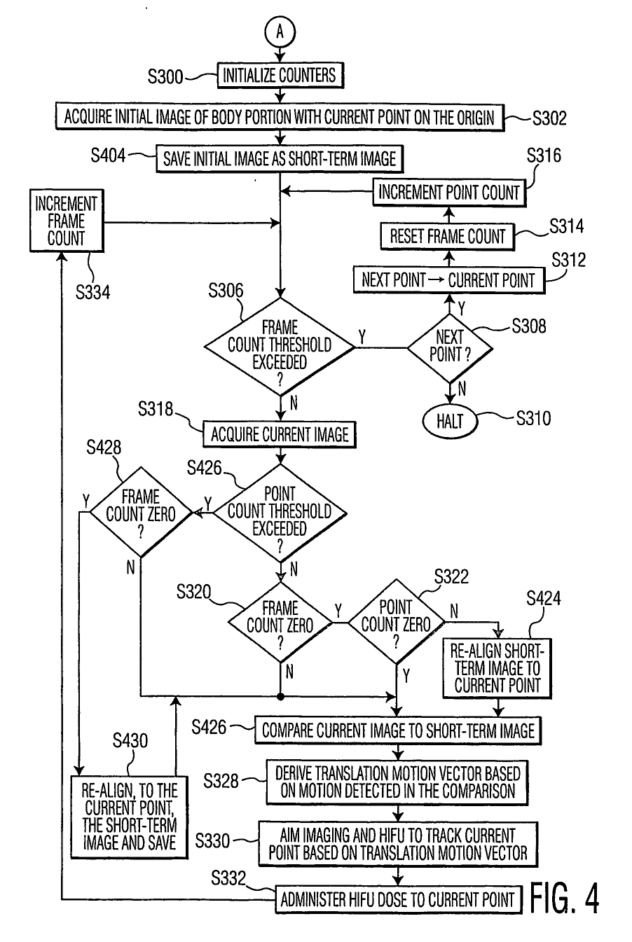

FIG. 4 shows a flow diagram of a second embodiment of an exemplary operational phase of a method of HIFU treatment according to the present invention.

FIG. 1 shows, by way of an illustrative, non-limitative example, a HIFU apparatus 110 for medically treating a patient in accordance with the present invention. The apparatus 110 includes an ultrasonic imaging system 112, a HIFU processor 113 and a robot arm 114 that is connected at its proximal end to the HIFU processor 113. At the distal end of the robot arm 114, the apparatus 110 further includes a HIFU transmitter 116, and a three-dimensional or “3-D” ultrasonic imaging transceiver 118 that emits ultrasound and receives back echoed ultrasound from which to form a 3-D image.

The HIFU processor 113 houses a controller 120 for operating the robot arm 114. In one embodiment of the invention, the controller 120 is a servo mechanism that is configured for precisely translating the robot arm 114 in any one or combination of three directions indicated in FIG. 1 by the axes x, y and z. Robot arm 114 can therefore move longitudinally forward and backward, horizontally left and right, and vertically up and down. The HIFU processor 113 uses a communication link 115 to communicate with the ultrasonic imaging system 112 prior to treatment in forming markers and during treatment in delivering HIFU dosage.

The ultrasonic imaging system 112 includes a real-time imaging processor 121 and an auxiliary processor 122. Leading from the real-time imaging processor 121, the ultrasonic imaging system 112 further includes a data bus 123, and on the data bus, a frame unit 124, a frame buffer 126, a frame counter 128, a point counter 130 and a timer 132. The frame unit 124 is configured for acquiring a succession of 3-D image frames from the transceiver 118 based on the received ultrasound and for storing the images in the frame buffer 126. As used herein, the term “3-D image frame” or “3-D frame” refers to an acquired set of ultrasonic images representing a 3-D volume. Since all frames discussed herein are 3-D frames, any reference a “frame” implies a “3-D frame.” The frame and point counters 128, 130 are used by the apparatus 110 in shifting treatment from one tumor spot to another. The timer 132 is used to regulate a duty cycle of the alternating imaging and HIFU transmission. The real-time imaging processor 121 acquires images and performs motion tracking. The processor 121 controls operation of its various components via signaling over the bus 123 and typically includes volatile and non-volatile memory such as read-only memory (ROM) and random-access memory (RAM) in any of their various forms.

The auxiliary processor 122 outputs imaging to a display 136 and has, as an input device 138, one or more of a mouse, joystick, keyboard, trackball or other known and suitable means of input. The display 136 and the input device 138 are operated to designate high-contrast ultrasonic markers and treatment volume boundaries prior to treatment and to initiate automatic treatment by the apparatus 110. The auxiliary processor 122 uses a communication link 133 to transmit the determined markers and boundaries and commands that initiate automatic treatment to the real-time imaging processor 121 or may transmit directly to the HIFU processor 113 over communication link 115.

The HIFU transmitter 116 includes a dish 140 that is typically 6 to 12 inches in diameter and houses at least one transducer element 142. A HIFU transducer element 142, shown on the underside of dish 140, surrounds the central hole of the HIFU transmitter 116. Although only one transducer element 142 is shown, multiple HIFU transducer elements 142 can be arranged in a configuration to surround the central hole. The ultrasonic imaging transceiver 118 generally comprises multiple imaging transducer elements (not shown). In an embodiment that is portrayed in the drawings, the transceiver 118 can be implemented with any known type of ultrasonic transducers suitable for 3-D imaging.

The imaging transceiver 118 and the HIFU transmitter 116 are both preferably mounted in fixed relative orientation so that, at all times, the imaging is disposed to acquire a three-dimensional image whose center coincides with the point where the HIFU would focus if HIFU were active, i.e., being transmitted—HIFU is preferably not active during imaging, because the HIFU sound waves would likely overwhelm the imaging. It is further preferable to fix both the transmitter 116 and transceiver 118 immovably to the robot arm 114, and to keep the HIFU beam invariable in focusing depth and orientation so that it never changes focus relative to the robot arm. Accordingly, the locations at which the HIFU is focused are totally and exclusively controlled by movement of the robot arm 114.

The invention is not, however, limited to implementation of the transmitter 116 and transceiver 118 in the above configuration. The HIFU transmitter 116 may, for example, be implemented with phased-array transducer elements, the electrical excitations to which are phased to steer the HIFU beam. Alternatively, a moving arm and a moving beam may be combined. As a further alternative, the robot arm 114 may tilt the dish 140 to a desired degree in one or both of two orthogonal directions, such as around the x and y axes, to provide an oblique angle for easy access to certain parts of the body. Furthermore, the transmitter 116 and the transceiver 118 may be disposed on the robot arm 114 asymmetrically, or may even be driven on different platforms for synchronized operation in treating the tumor.

Also depicted in FIG. 1 is a schematic cross-sectional drawing of a torso 144 of the medical patient to be treated using HIFU. The patient is shown lying down, face up, as indicated by the orientation of the ribs 146 and the connecting spine 148, although the patient could be positioned otherwise. In proximity of the patient's skin 150 is a container 152 which is filled with a liquid, such as water, that is utilized to transmit the HIFU to the patient in a conventional manner. Within the torso 144 are organs or other body portions 162, 164. The body portion 164 contains a treatment volume 166, which, in turn surrounds a tumor 168. The HIFU is shown as a beam 158 focused on a point 176 within the tumor 168.

The three-dimensional field-of-view of the imaging, part of which is delimited by the dotted lines 169, is configured large enough to account for off-center movement of the tumor that may occur in between successive motion compensations.

Formed or anchored within the body portion 164 are three ultrasonically high-contrast markers 170, 172, 174, although fewer or more markers may be utilized. The markers 170, 172, 174 are preferably formed by applying HIFU to “burn” them in, although markers may be implemented by injection of ultrasound contrast agents or by implantation of pellets or pins of a biocompatible material, for example. The markers 170, 172, 174 are not always needed, depending upon the visibility or contrast of the tumor 168 against surrounding tissue. Markers can be formed inside or outside of the tumor 168. Tissue will regenerate in the liver and small losses of tissue in the breast are not detrimental, so that markers can be formed outside of tumors for these organs. Whether formed within the tumor 168 or merely within the treatment volume 166 or body portion 164, markers should be positioned to avoid being obscured by treatment for the entire treatment or for as long as feasible during the treatment. Thus, since HIFU irradiation of the treatment volume 166 at point 176, especially via cooking, generally blocks subsequent visibility of tissue behind the point treated, treatment begins toward the portion of the treatment volume's rear, as seen from the HIFU transducer, denoted by line R. Markers are preferably formed outside of the tumor, if feasible, as with markers 170, 172, 174 or in front of or towards the front of the tumor as with markers 170, 174.

Shown in FIG. 2 is a flow diagram of an exemplary preparatory phase of the invention in which the doctor first views on the display 136 an image of the body portion 164 that is to be treated (step S200). If the tumor 168 is close to the patient's heart and therefore moves with the patient's heart beat, the marker burned in by HIFU may miss its intended location point. Since there is flexibility in locating the markers for motion tracking, the marker will generally still be useful. However, its use may require a rethinking of the treatment volume boundaries. For example, non-tumorous tissue in the treatment volume may be in front of the marker, but could be excluded from a redrawn volume. It is accordingly preferred that markers 170, 172, 174 be formed before defining the treatment volume 166.

The doctor maneuvers the input device 138 and, correspondingly, the screen cursor over the image of the body portion 164 and further manipulates the input device 138 to designate a marker (step S202). Automatically, or through further operation of the input device 138 or other input means, HIFU is transmitted to focus on the designated point to create a marker at that point (step S204). The doctor views the marker(s) created (step S206) and decides whether to place another marker (S208). If another marker is to be formed, the process repeats starting at step S202 until the last marker has been burned into the body portion 164.

The doctor next defines the treatment volume 166 by maneuvering a mouse 138, joystick or other input device so that an overlay visible on the display 136 delimits the treatment volume boundaries.

The points within the treatment volume 166 are then accorded an order in which they are to be dosed. In a preferred embodiment of the invention, the points are treated in raster scan order, e.g., from left to right and from top to bottom, starting at the back of the treatment volume 166 in the plane or slice that contains line R in FIG. 1, and proceeding frontward plane by plane. Motion tracking compensates for any slanting of the treatment volume 166 that may occur during treatment, as a result of a heart beat, breath, or other event, so that raster order is maintained

The physician places the screen cursor at the point within the treatment volume at which the raster scan is to begin, or, in an especially preferred embodiment of the invention, the apparatus 10 automatically designates the first point in the raster (step S210). Raster order is not the only possible ordering, however, and the operator may use the input device 138 to designate another ordering instead.

FIG. 3 shows a flow diagram of a first embodiment of an exemplary operational phase of a method of HIFU treatment for those situations where at least one marker has been formed outside of the treatment volume and therefore will not be obscured by the treatment. The first embodiment always compares the current image to the initial image to determine motion compensation, where the initial image is the image that was acquired at the very beginning of treatment. This technique minimizes accumulated error and is made feasible by the existence of markers outside the treatment volume.

Motion tracking, whether in two or in three dimensions, typically involves rotating and/or translating an image in a first frame, overlaying the moved image on a second frame, obtaining a correlation between the two images, and repeating the process, each time using a different increment for rotation and/or translation. The total rotation and/or translation associated with the highest correlation represents the motion to be compensated. That rotation and/or translation is said to bring the images into “registration” so that pattern recognition has occurred. For three-dimensional tracking, the compensation that bring the two images into registration is expressed as a six-dimensional vector, e.g. corresponding to increments for rotation about the x, y and z axis and increments for translation in the x, y and z directions.

Hypothetically, frame comparisons to detect motion of the body portion 164 could be performed using strictly-time-adjacent frames, i.e., a frame and the next frame. In the interim period between acquisition of the frames, only one point in the treatment volume 166 has been dosed, making the images to be compared very similar. Thus, the entirety of the image can be subject to motion tracking, aiding in the attainment of registration.

However, a disadvantage of comparing strictly-time-adjacent frames is that errors accumulate frame-to-frame in aiming the imaging transceiver 118 to compensate for motion. Error may arise, for example, in the magnitude of the determined motion compensation along one or more of the three axes x, y and z, or in the distance that the servo 120 moves the robot arm 110 along one or more of the three axes to perform the determined motion compensation. The error manifests in an imperfect tracking of the point that is to receive HIFU dosage. Any translation bias in the determined motion compensation and/or in the responsive translation commands to the servo 120 will accumulate. If the biases are random, they will tend to cancel out over the long run but will still produce sizable deviations at times. If, on the other hand, the biases are systematically in one direction, errors will accumulate even quicker.

By contrast, errors do not accumulate if the current image is compared to the initial image, as in the first embodiment of the present invention. The center or “origin” of the initial image is directly on the first point of the raster, whether or not it was the operator or the apparatus 10 that, by automatic operation, designated the first raster point in step S302. The inventive technique of the first embodiment shifts the initial image so that its origin coincides, instead, with the current point and then compares the shifted initial image to the current image to determine motion compensation. The HIFU transmitter 116 and the imaging transceiver 118 are then translated by the robot arm 114 based on the determination.

Referring again to FIG. 3, the frame and point counts are initialized by resetting the corresponding counters 128, 130 (step S300). An initial image of the body portion 164 is acquired with the origin located on the first treatment point to be HIFU dosed (step S302). Therefore, for example, once the first raster point is designated (step S210) on the display 136 via the mouse 138, imaging shifts to center that first raster point onto the origin. The initial image is then saved to memory (step S304).

At this juncture in the processing, a determination is made as to whether a predetermined frame count threshold has been exceeded (step S306). The frame count serves as timing mechanism by which the HIFU transmitter 116 delivers a proper dose to the current point in the body portion 164. Images are acquired at a frame rate of preferably at least 2 frames per second, with a duty cycle of, for example, 20%. Thus, at 2 frames per second, an imaging time period of 0.1 seconds is followed by a HIFU time period of 0.4 seconds, which, in turn, is followed by another 0.1 seconds of imaging, and so on, in interleaved fashion. Once a predetermined number of frames have been acquired, driving the frame count to a predetermined magnitude, it follows that a predetermined number of HIFU doses have been administered. A frame count threshold is therefore established, whereby exceeding of the threshold indicates that dosage has been completed for the current point. Timing mechanisms other than a frame count can be employed instead. It would also be possible to provide feedback imaging by which an assessment, according to specified criteria, could be made that dosage for the current point is complete. An example would be MRI feedback based on temperature at the current point, although one advantage of the present invention is the opportunity to break free of MRI cost overhead. In that case, when it is determined that dosage has been completed for the current point, the ultrasonic imaging system 112 could issue a command or other indicator to the HIFU processor 113 to immediately terminate the current HIFU dosage cycle.

Next, query is made as to whether a next point exists in the raster (step S308). If not, therapy is done and the operational phase halts (step S310). If, on the other hand, a next point exists, that next point is made the current point for subsequent processing purposes (step S312), the frame count is reset (step S314) and the point count is incremented (step S316).

If the frame count threshold has not been exceeded (step S306), the ultrasound imaging is restarted, with its current aiming, to acquire a current image (step S318). Thus, for example, if this is the first iteration of step S318, imaging has not been re-aimed; on the other hand, if this not the first iteration of step S318, the imaging may have been re-aimed since the most recent previous iteration of step S318.

Query is next made as to whether the frame count is zero (step S320). If so, and if the point count in non-zero (step S322), a next point has been selected as the current point (step S312) but motion tracking has not yet occurred for that current point. To prepare for the comparison of images in motion tracking, the initial image is re-aligned so that its origin coincides with that current point (step S324). On the other hand, is the frame count is non-zero or if the point count is zero, re-alignment is not needed.

Next, the current image is compared to the initial image (step S326), which has or has not been re-aligned as described above. Any difference between the two images being compared is attributable either to motion of the body portion 164 or to re-alignment of the initial image for the next point or to both. The motion-tracking algorithm will output a six-dimensional vector that reflects this difference and will, for simplicity, be regarded hereinafter as a motion vector or motion compensation vector.

The six-dimensional motion compensation vector is preferably arranged so that the three rotations precede the three translations. Each rotation or translation can be described by a matrix, so that the six matrices are multiplied in a predetermined order. Matrix multiplication, however, is not commutative, i.e. matrix A time matrix B does not generally equal matrix B times matrix A. If the determined motion compensation is expressed so that the rotations precede the translations, the rotations can be ignored. That is, since the current point is on the origin, rotations with respect to any of the three axes x, y, z do not move the current point Therefore, the only components of the determined motion compensation that are needed are the three translations, which comprise a three-dimensional translation motion vector (step S328).

Each translation corresponds to a respective translation by servo 120 of the robot arm 114 in the respective direction. Moving the robot arm 114 accordingly aims the imaging and the HIFU to track the current point (step S330). If no motion or initial image re-alignment has occurred, the translation motion vector entries are zero and the robot arm 114 is kept stationary.

If the tumor to be treated is so located on the patient's body that general anesthesia is not required, it is possible that the patient may move sufficiently to cause the tumor 168 to leave the imaging field-of-view, and the current image, to a degree that the motion tracking algorithm fails to register the current image with a previous image. In that case, HIFU transmission cannot continue, because of possible stray dosage to the patient, so processing is halted (not shown).

After the aiming in step S330, a HIFU dose is administered to the current point (step S332), the frame count is incremented (step S334), and the image acquisition phase is repeated if dosage for the current point is not completed or if it is completed and a next point exists.

FIG. 4 shows a second embodiment of an exemplary operational phase of a method of HIFU treatment for those situations not covered by the first embodiment, i.e., where no markers have been formed or none have been formed outside the treatment volume 166. Any markers can only be utilized for motion tracking so long as they remain unobscured by treatment. Once the markers are obscured, or if no markers were formed, motion tracking can only rely on the treatment volume 166. As the treatment volume 166 is progressively treated, however, the treatment volume 166 in the current image differs more and more from the treatment volume 166 in the initial image. Motion tracking is therefore made to rely on only that portion of the treatment volume 166 that has not yet been treated and therefore resembles the corresponding portion of the treatment volume 166 in the initial image. Since only part of the treatment volume 166 is being registered between the two images, registration becomes progressively more unstable. At some stage of the treatment, therefore, the present invention compares the current image not with the initial image but with a more recently-acquired image. Advantageously, the present invention methodology accomplishes this shift in technique without any significant accumulation of error, by comparing each subsequent image to the first-acquired image for that treatment point.

In FIG. 4, steps S300 to S318 carry over unchanged from FIG. 3, except that step S404 not only saves the initial image, but saves it as a “short-term” image. A short-term image is utilized only for the current point under HIFU treatment.

After step S318, query is made as to whether the point count threshold has been exceeded. If not, all the succeeding flow chart steps from FIG. 3 carry over, except that step S424 re-aligns, to the current point, the short-term image, rather than the initial image, and step S426 compares the current image to the short-term image, rather than to the initial image.

If the point count threshold is exceeded (step S426), query is made as to whether the frame count is zero (step S428). If so, the short-term image is re-aligned to the current point, saved as the “new” short-term image (step S430), step S426 is executed and processing proceeds with steps S328 through S334 to complete an iteration for a frame. If the frame count is not zero, step S426 is likewise executed and processing proceeds, in like manner, with steps S328 through S334 to complete an iteration for a frame.

The point count threshold is selected so that when it is exceeded, the number of points in the treatment volume 166 that have, by that time, been dosed is sufficient so that comparing the current image to the initial image (step S426) is foregone henceforth in favor of comparing the current image to a more recently-acquired image (step S430). An appropriate point count threshold can be determined based on empirical data.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method for medically treating a patient comprising the steps of:
a) using imaging ultrasound to acquire an initial image of a portion of the patient's body; and
b) performing, a predetermined non-zero number of times, the steps of:
i) using imaging ultrasound to acquire a current image of the patient's body portion;
ii) comparing said current image to a previously-acquired imaging ultrasound image;
iii) detecting, based on the comparison, whether the body portion has moved since the previously-acquired image was acquired;
iv) if motion is not detected, administering a dose of high-intensity focused ultrasound (HIFU) to a point in the body portion; and,
v) if motion is detected, characterizing the motion, aiming a HIFU transmitter based on the characterization to track said point and administering said dose to said point.

2. The method of claim 1, wherein at least steps ii) through v) are performed under automatic processor control and without user intervention.

3. The method of claim 1, wherein step v) further comprises the step of aiming the imaging ultrasound to track said point.

4. The method of claim 1, wherein steps i) through v) are performed a plurality of times and the current images are acquired at a rate of at least two current images per second.

5. The method of claim 1 wherein said images are representative of respective three-dimensional volumes and are represented by respective three-dimensional image frames, the method further comprising, before step b), the steps of setting a three-dimensional image frame count to zero and determining and ordering a set of points in the body portion upon which to focus transmission of HIFU, wherein step b) further comprises, for each iteration, the steps of:
determining whether the frame count exceeds a predetermined frame count threshold;
if the frame count threshold has not been exceeded, incrementing the frame count; and
if the frame count threshold has been exceeded:
determining whether a next point in the ordered set exists;
if a next point exists, resetting the frame count to zero and using said next point as said point in the body portion in steps iv) and v); and,
if a next point does not exist, halting further performance of steps i) through v).

6. The method of claim 5, wherein aiming the HIFU transmitter to track a point in step v) also aims the imaging ultrasound to track that same point and does not cause the HIFU to change focus.

7. The method of claim 5, wherein, after a predetermined number of points have been HIFU dosed in step v), said previously-acquired imaging ultrasound image in step ii) is the image first acquired in step i) after the frame count threshold was last exceeded.

8. The method of step 1, wherein, for at least one step i) through v) iteration after the first iteration, said previously-acquired imaging ultrasound image in step ii) is the initial image acquired in step a).

9. The method of claim 1, further including, before step b), the step of aiming said HIFU transmitter at said point.

10. The method of claim 1, further including the step of using HIFU to place in the body portion at least one ultrasonically high-contrast marker for use in making the comparison in step ii).

11. An apparatus for medically treating a patient, comprising:
an ultrasonic transceiver for emitting and receiving ultrasound to image a portion of the patient's body;
a frame buffer;
a frame unit for acquiring a succession of image frames from the ultrasonic transceiver based on the received ultrasound and for storing the succession of image frames in the frame buffer, each image frame constituting an acquired set of ultrasonic images representing a 3-D volume;
a processor for comparing image frames in the frame buffer to detect motion of the body portion;
a high-intensity focused ultrasound (HIFU) transmitter operable to focus on a point in the body portion; and
a controller for causing transmission from the HIFU transmitter to track said point if said motion is detected by said processor.

12. The apparatus of claim 11, further including a timer, wherein the processor is configured to alternate, based on expiry of the timer, image frame acquisitions by the frame unit and transmission by the HIFU transmitter so that a transmission follows an image frame acquisition and vice versa.

13. The apparatus of claim 12, further including a counter for counting image frame acquisitions, said processor being configured to halt HIFU transmission to said point when the counter reaches a predetermined count.

14. The apparatus of claim 13, wherein:
said HIFU transmitter is operable to focus on a predetermined set of ordered points in the body portion;
said processor is further configured to determine, based on a current one of the ordered points, whether a next point in the set exists; and
said controller is operable to cause the HIFU transmitter to track said next point if said next point exists and if said processor has detected motion of the body portion.

15. The apparatus of claim 12, wherein the frame unit is configured to acquire image frames at a rate of at least 2 frames per second.

16. The apparatus of claim 11, wherein said controller is further configured to aim the ultrasonic transceiver to track said point if motion is detected by said processor.

17. The apparatus of claim 16, further including a robot arm that is connected to the ultrasonic transceiver and the HIFU transmitter and is operable by said controller.

18. The apparatus of claim 17, wherein the HIFU transmitter is configured with a central hole that contains the ultrasonic transceiver.

19. The apparatus of claim 18, wherein the ultrasonic transceiver is mounted in fixed relative orientation to the HIFU transmitter.

20. The apparatus of claim 11, further including a user-operable input device for defining boundaries of a treatment volume within said body portion, said point residing within said treatment volume, and for defining at least one ultrasonically high-contrast marker for use in said comparing.

21. The apparatus of claim 11, wherein said controller halts HIFU processing when it receives an externally supplied indicator that sufficient dosage has been applied.

22. The apparatus of claim 11, wherein said controller halts HIFU processing when it receives an ultrasound image based indicator that sufficient dosage has been applied.
